(19) United States
(12) Patent Application Publication
(10) Pub. No.: US 2014/0323912 A1
(43) Pub. Date: Oct. 30, 2014

(54) SYSTEM AND METHOD FOR IDENTIFYING A CONDITION OF PELVIC FLOOR MUSCLES

(71) Applicant: The Procter & Gamble Company, Cincinnati, OH (US)

(72) Inventors: Ryo MINOGUCHI, Cincinnati, OH (US); Diana Lynne GANN, Lebanon, OH (US); Alexzandra Joan SPATHOLT, Cincinnati, OH (US)

(73) Assignee: The Procter & Gamble Company, Cincinnati, OH (US)

(21) Appl. No.: 14/219,103

(22) Filed: Mar. 19, 2014

Related U.S. Application Data
(60) Provisional application No. 61/817,011, filed on Apr. 29, 2013.

(51) Int. Cl. A61B 5/20

(52) U.S. Cl. CPC A61B 5/207
USPC 600/574

(57) ABSTRACT

Embodiments disclosed herein are configured for identifying a condition of pelvic floor muscles. In one embodiment, a kit is provided that includes a wetness detection device with a backsheet, where the backsheet is substantially impervious to liquid. The wetness detection device may additionally include an insert that is coupled to the backsheet via a securing mechanism, where the insert is constructed with a predetermined absorbency. The wetness detection device may include an indicator that is coupled to the insert that reacts when in contact with liquid, such that because of the predetermined absorbency of the insert. The indicator may identify progress on pelvic floor muscles of a wearer of the wetness detection device. Additionally, the kit may include instructions for exercising the pelvic floor muscles to perform while wearing the wetness detection device, where the instructions describe application of the wetness detection device.

### BACKGROUND OF THE INVENTION

Urinary incontinence is a problem that decreases the quality of life for many women. The problem is generally caused by lack of balance between the bladder pressure and the urethral closure pressure at rest and/or during activities such as coughing, sneezing, laughing, hiccupping, tightening, bearing down, walking, running, etc. This imbalance is due to deficiency of mechanical function (referred as stress urinary incontinence) and/or neurological function (referred as urge urinary incontinence or overreacting bladder) in controlling the muscles in the pelvic floor associated with bladder action and urethral closure.

Various techniques may be utilized for improving functions of the muscles in the pelvic floor to decrease urine leakage. Amongst them, one called the KNACK has been disclosed as an effective therapy focusing on learning timely and correct contraction of the muscles in the pelvic floor. This technique may utilize a feedback device to quantify the amount of urine leakage in diagnosis at the start of the training and in monitoring the progress of the training. A brown paper towel has been disclosed for the purpose, which is used being held by a woman against a pudendal region of her body and provides visual feedback on the amount of urine leakage during an intentional cough. Because the paper towel needs to be held against the pudendal region of the body, this technique can be practiced only in private spaces such as a physician's office, a physiotherapist's office, a bathroom, etc., during limited times of a day. Further, a brown paper towel can allow urine to penetrate through and wet her hand as she attempts the procedure. Such a scenario also can cause discomfort by placing a paper towel against a sensitive region of the woman's body. Additionally, such a configuration cannot accommodate unexpected heavy flow during practicing the technique.

### SUMMARY OF THE INVENTION

Embodiments of the disclosed herein include a kit with a wetness detection device and instructions. The wetness detection device may include an insert with an indicator for detecting liquid that can be placed on top of the wetness detection device or be incorporated within the wetness detection device during manufacturing. The instructions may be utilized for communicating a self-diagnostic test procedure for assessing the value of attempting a pelvic training and/or for providing training for performing the training exercises.

Similarly, some embodiments include a standalone wetness detection device and/or insert. The insert may be configured with predetermined absorbent characteristics and a liquid indicator that allows spread of liquid to determine volume of the liquid. Depending on the particular embodiment, the insert may be coupled to an exterior portion of a wetness detection device and/or incorporated within the wetness detection device. The insert may also be coupled or otherwise associated with a user's undergarments. The wetness detection device or insert alone can be coupled to a separately manufactured and purchased absorbent article, such as, for example, an incontinence device or menstrual product.

### BRIEF DESCRIPTION OF THE DRAWINGS

FIG. 1 depicts a wetness detection device for determining a volume of liquid through a linear measurement of liquid sprawl on a continuous indicator on an insert, according to embodiments disclosed herein;

FIG. 2 depicts a cross sectional view of a wetness detection device for determining a volume of a liquid through a linear measurement of liquid on an indicator that is integrated into the wetness detection device, according to embodiments disclosed herein;

FIG. 3 depicts a wetness detection device for determining a volume of liquid through a linear measurement of the liquid on a plurality of rectangular indicators, according to embodiments disclosed herein;

FIG. 4 depicts a wetness detection device for determining a volume of a liquid through a linear measurement of liquid on a plurality of rounded indicators, according to embodiments disclosed herein;

FIG. 5 depicts a cross sectional view of a wetness detection device determining a volume of a liquid through a linear measurement of liquid on an indicator that is applied to an outer surface of the wetness detection device, according to embodiments disclosed herein;

FIG. 6 depicts a wetness detection device for determining a volume of a liquid through a linear measurement of the liquid on a continuous indicator applied to the wetness detection device, according to embodiments disclosed herein;

FIG. 7 depicts a cross sectional view of a wetness detection device for determining a volume of a liquid through a linear measurement of the liquid on an insert of the wetness detection device, according to embodiments disclosed herein;

FIG. 8 depicts a flowchart for providing training for pelvic floor muscles for urinary incontinence, according to embodiments disclosed herein;

FIG. 9 depicts a flowchart for utilizing a training kit for identifying a condition of pelvic floor muscles, according to embodiments disclosed herein; and

FIG. 10 depicts a flowchart of a process that a wearer may utilize with the kit, according to embodiments described herein.

### DETAILED DESCRIPTION OF THE INVENTION

Embodiments disclosed herein include a wetness detection device with an insert that includes least one indicator that may be utilized for determining a volume of liquid. The wetness detection device may be part of a kit that also includes instructions for testing and training pelvic floor muscles of a wearer. The insert can be placed on top of the wetness detection device and/or may be incorporated within the wetness detection device. Depending on the particular embodiment, placement of the insert may be performed during manufacturing and/or by a consumer after purchase. The indicator may be configured for quantifying the volume of liquid absorbed by each insert. The indicator is described herein as part of an “insert”; however, an indicator may simply be printed or otherwise integrally included on other features of a wetness detection device.

The instructions that are included in the kit may be utilized for a health provider-assisted and/or a self-diagnostic test procedure. The test procedure includes contracting one's pelvic floor muscles while applying downward pressure on one's abdomen to assess the amount of urine leakage during a stress induced incontinence episode in which the pelvic floor muscles are tightened to enhance the restriction force of the urethra sphincter muscles. The instructions may additionally describe exercises for strengthening the pelvic floor muscles, as well as subsequent testing procedures.

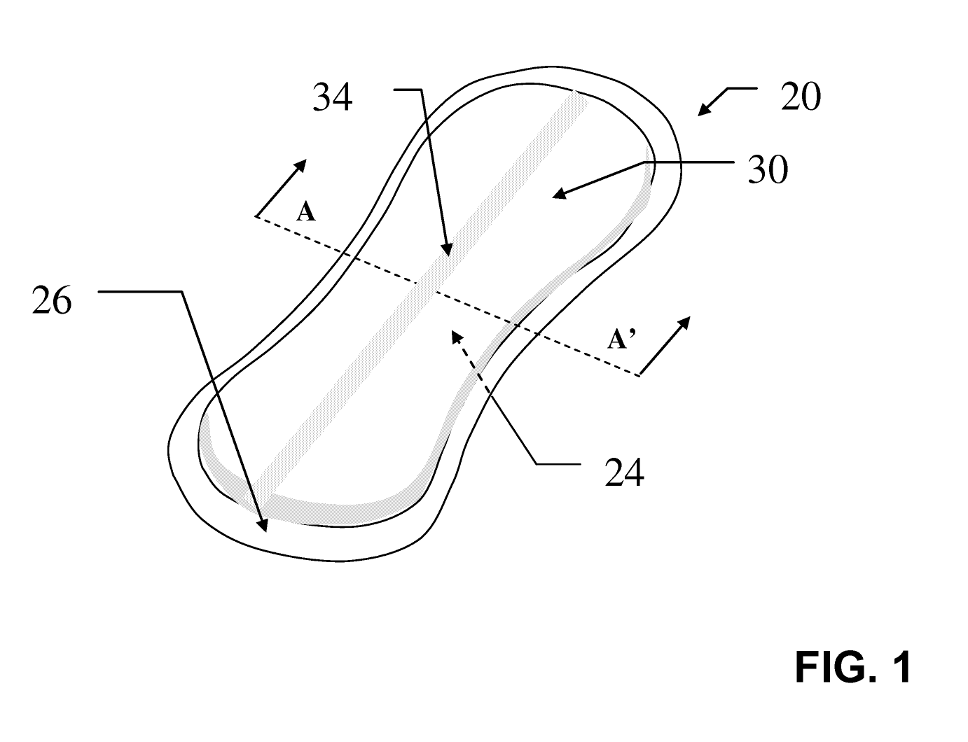

Referring specifically to the drawings, FIG. 1 depicts a wetness detection device 20 for determining a volume of liquid through a linear measurement of liquid on an indicator 34 on an insert 30, according to embodiments disclosed herein. The wetness detection device 20 may be configured in a wide variety of sizes and shapes, such as oval, rectangle, “dog bone,” “hourglass,” etc. and may include the insert 30 that defines a body facing surface and a backsheet 24 that defines a garment facing surface that is opposed to the body facing surface. As illustrated in FIG. 1, the insert 30 may function as a top sheet (such as a primary top sheet and/or a secondary top sheet), absorbent article, etc. The insert 30 also includes an indicator 34 along a longitudinal centerline through entire length of the wetness detection device 20. The indicator 34 is depicted in FIG. 1 as a continuous indicator in that a single strip indicator is depicted. As described in more detail below, this is merely an example, as other configurations of indicators may be utilized. Insert 30 and/or indicator 34 can alternatively be associated with any layers below the device topsheet so long as indicator 34 is visible through the topsheet.

As the insert 30 will likely be placed against the wearer's skin, the insert 30 may be compliant, soft feeling, and non-irritating. The insert 30 may include a portion that is (or may entirely be) liquid pervious or permeable, permitting liquid bodily exudates (e.g., urine, menses, etc.) to readily penetrate through its thickness. Liquid pervious materials may be manufactured from a wide range of materials such as woven and nonwoven materials (such as hydrophilic or rendered hydrophilic materials); polymeric materials such as thermoplastic films, porous thermoplastic films, apertured thermoplastic films, textured thermoplastic films; foam materials such as porous foam materials, reticulated foam materials; etc. Suitable woven or nonwoven materials can include natural fibers (e.g., pulp, cotton fibers, cotton linters), regenerated natural fibers (e.g., rayon fibers, viscose rayon fibers, cupper ammonium rayon fibers, cellulose acetate fibers), synthetic fibers (e.g., polymeric fibers such as polyester fibers, polypropylene fibers, polyethylene fibers) or from a combination of the fibers.

The insert 30 may include textures, folds, holes, apertures, slits, and any other mechanical manipulation to alter physical characteristics and appearance of materials of which the insert is made, such as coefficient of friction, softness, flexibility, resiliency, permeability, etc. In some embodiments, the insert 30 can include color, graphics, prints, etc.

The backsheet 24 may be substantially impervious to liquid and may be manufactured from a thin plastic film, although other flexible liquid impervious materials may also be used such as a liquid impervious but vapor pervious film/nonwoven laminate, a microporous film, an apertured formed film, or other polymer film that is vapor permeable, or rendered to be vapor permeable, but substantially impervious to fluid. As such, the backsheet 24 may be constructed of materials that are compliant and will readily conform to the general shape and contours of the human body. The backsheet 24 may be configured to prevent the liquid bodily exudates absorbed and contained in the absorbent core 60 from wetting articles that contact the wetness detection device 20. The backsheet 24 may thus include a woven or nonwoven material, polymeric films such as thermoplastic films of polyethylene polypropylene, composite materials such as a film-coated nonwoven material, and/or other similar materials.

Some embodiments may be configured with the insert 30 joined to the absorbent core 60 (and/or the secondary top sheet if present) by a fusion bond resulting from an application of heat/pressure. The pattern of the fusion bond can include a continuous line(s) and/or a number of discrete portion of fusion bonds.

Further, the backsheet 24 may be provided with adhesive fasteners for attaching the wetness detection device 20 to the wearer's undergarment. Wetness detective device 20 may employ lateral flaps or wings that can be wrapped around the wearer's undergarment for a more secure fit. The garment facing surface of the flaps, adjacent the distal edges of the flaps, may also be provided with a flap adhesive fastener.

As described above, the wetness detection device 20 can be (or can include) any kind of external absorbent article, such as a panty liner, menstrual pad, or incontinence product.

The wetness detection device 20 may be configured to absorb liquid bodily exudates such as sweat, blood, urine, menses, etc. The wetness detection device 20 may be disposable and thus may be partly or wholly disposed of after a single use.

It should be understood that the wetness detection device 20 can generally have any thickness including relatively thick, intermediate thickness, relatively thin, or even very thin (or “ultra-thin”). Embodiments of “ultra-thin” wetness detection devices include a caliper of less than about 3 mm. Based on the volume of liquid that has been received by the wetness detection device 20, a recommendation may be made regarding the thickness of the next wetness detection device that the wearer may purchase.

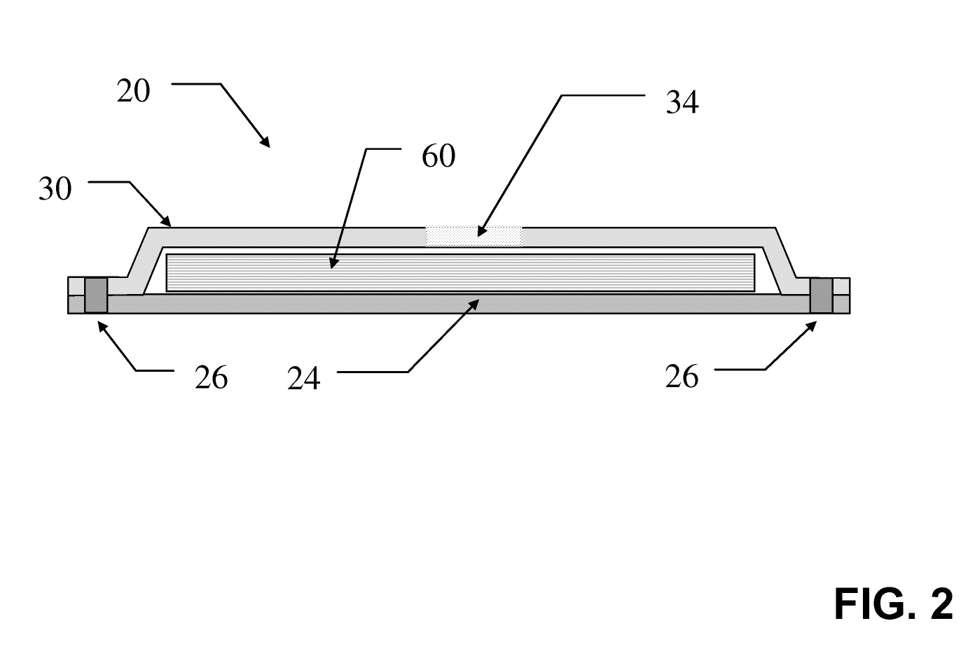

FIG. 2 depicts a cross sectional view along line A-A′ of the wetness detection device 20 from FIG. 1 for determining a volume of a liquid through a linear measurement of liquid on an indicator 34 that is integrated into the wetness detection device, according to embodiments disclosed herein. As discussed above, the wetness detection device 20 may include an insert 30 (which may be liquid pervious), a backsheet 24 (which may be liquid impervious), and an optional absorbent core 60 disposed between the insert 30 and the backsheet 24. The absorbent core 60 may be configured for receiving, absorbing, and/or retaining a liquid bodily exudate. The absorbent core 60 may be compressible and/or conformable. The absorbent core 60 may include a single layer or a plurality of layers, depending on the particular embodiment. The absorbent core 60 may have a wide variety of sizes and shapes, at least a portion of which conform to the shape of the wetness detection device 20 as a whole. The absorbent core 60 may include absorbent materials such as woven materials, nonwoven materials (e.g., wetlaid tissue, airlaid materials, carded materials, fluff materials, spunlace materials, spunbond materials, etc.), foams, sponges, porous beads, super absorbent polymers, and the like.

As also illustrated in FIG. 2, the insert 30 and the backsheet 24 may each cover a primary surface of the absorbent core 60 and may be secured together with one or more securing mechanisms 26 to fully enclose the absorbent core 60. The insert 30 and the backsheet 24 may be permanently or temporarily secured together, such as with adhesive (e.g., tape, glue, etc.), bindings (e.g., staples, clamps, etc.), and/or other securing mechanisms. The insert 30 and the backsheet 24 preferably extend in both the longitudinal and transverse directions to reach the circumferential edge of the wetness detection device 20. In some embodiments, the insert 30 and backsheet 24 are densified by application of pressure or heat and pressure to form a crimp seal.

In operation, the insert 30 may be configured to absorb liquid bodily exudates such as sweat, urine, blood, menses, etc., and distribute the liquid bodily exudates on and/or under its surface. In one embodiment, the indicator 34 is placed on top of the wetness detection device 20 by a wearer during the self-diagnostic test procedure. In another embodiment, the indicator 34 is incorporated within the wetness detection device 20 during manufacturing of the wetness detection device.

In embodiments where the insert 30 is a material or a product being placed on top of the wetness detection device 20, the insert 30 may include adhesive on one surface so that the insert 30 may be substantially secured to the wetness detection device 20 while being worn by the wearer. The adhesive can be located on a portion of the insert 30 (such as along the perimeter) so that a liquid bodily exudate is absorbed by the insert 30 for being absorbed by the absorbent core 60. When the insert 30 is incorporated within the wetness detection device 20 during manufacture, the insert 30 can be configured as a top sheet or as a secondary top sheet of the wetness detection device 20.

The insert 30 may include a material that is wettable by a liquid bodily exudate such as urine and may exhibit a contact angle with a saline solution of less than 90 degrees. The insert 30 may include one or more macroscopic or microscopic spaces that allow a liquid bodily exudate such as urine to pass or flow through based on the surface tension of the fluid relative to the solid surfaces of the materials that compose the insert 30. The insert 30 may include of a single layer or a plurality of layers. The plurality of layers may be joined such as via adhesive, mechanical bonding, thermal bonding, and/or other joining mechanisms.

Additionally, the indicator 34 can change in appearance and/or provide a signal after being in contact with a liquid. The indicator 34 can indicate the amount of a liquid bodily exudate absorbed by the insert 30 through a linear measurement of the spread of liquid. Referring back to FIG. 1, the indicator 34 may be configured to change appearance or otherwise indicate at those portions of the indicator 34 that actually contact liquid (and/or a specific liquid). The change in appearance may be due to a chemical reaction between the indicator 34 and the liquid, an electrical reaction, and/or other reaction. Additionally, the insert 30 and absorbent core 60 are configured to provide a predetermined linear reach of the liquid, based on volume. As an example, the insert 30 may be configured with absorbent properties such that a tablespoon of liquid sprawls one-quarter inch on the surface of the insert 30. Thus, if a tablespoon of liquid were received by the insert 30, one-quarter inch of the indicator 34 would identify wetness. A determination may then be made regarding the volume of liquid received by the wetness detection device 20.

As discussed above, the indicator 34 may be configured to change color, change visibility, and/or send an electrical signal. For a color change, the initial color of the indicator 34 and the subsequent color can each be any variation of any color, so long as the subsequent color is visually distinguishable from the initial color. For an electrical signal, the indicator 34 may be coupled to an integrated circuit or other electrical device that identifies when and at which portions of the indicator 34 a liquid was received. Upon detecting the presence and location of the liquid, the integrated circuit may send a signal to a computing device with digital display, such as a personal computer, mobile device, server, etc. or otherwise communicate to a user (wearer, health care provider, etc.) the volume of liquid received by the wetness detection device 20.

Embodiments discussed above refer to identifying a volume of liquid based on a measurement of the sprawl of that liquid. This may be due to the predetermined absorbency of the insert 30 and the absorbent core 60. However, some liquids may have different properties and may thus behave differently when in contact with the wetness detection device 20. Accordingly, some embodiments may be configured to identify the type of liquid that has been received and, based on the type of liquid, identify the volume. As an example, if it is determined that the liquid is urine, the indicator 34 may change into a first color or provide a first signal type. Based on the color and the sprawl of the urine, a determination may be made regarding the volume of the liquid. If the liquid is menses, the indicator 34 may change into a second color or signal and a volume determination may be made based the sprawl of the liquid and based on the fact that menses may have a different sprawl than urine. In some embodiments, the indicator may only react to one type of liquid. Accordingly, the wetness detection device may be used to help a user quantify menses flow for selecting an appropriate menstrual product.

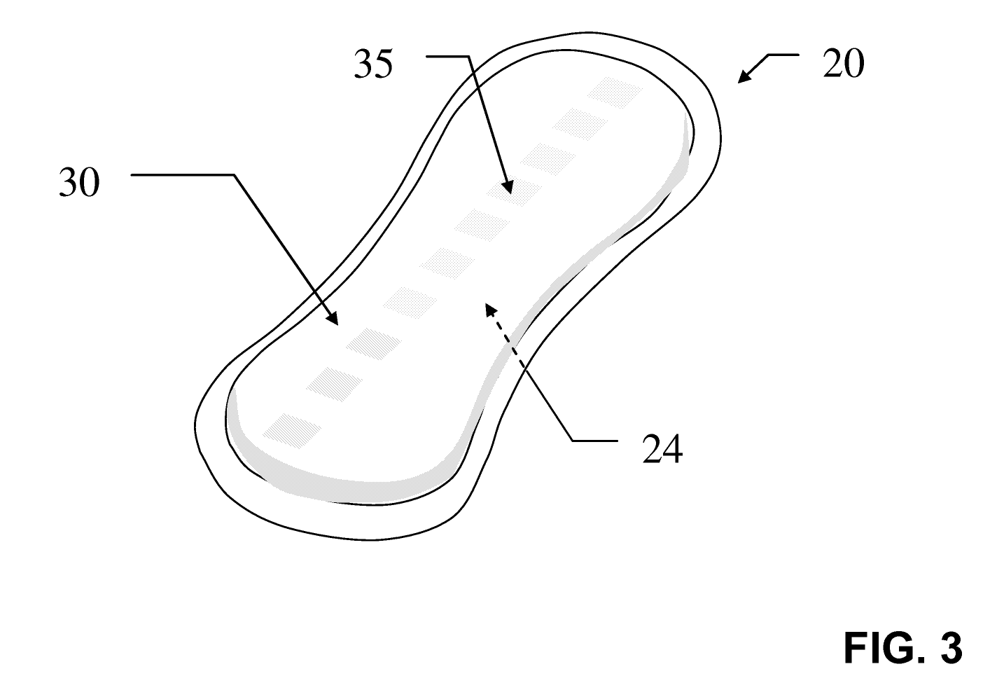

FIG. 3 depicts a wetness detection device 20 for determining a volume of liquid through a linear measurement of the liquid on a plurality of rectangular indicators 35, according to embodiments disclosed herein. As illustrated, the rectangular indicators 35 may include a series of lines or a grid pattern. The lines or grid patterns can further be coupled with a number system used for determining an area or length of the insert 30 that was exposed to the liquid. Determining an area or length the liquid sprawl can be as simple as counting, adding up, or reading the numbers at the two extremes of the areas exposed to wetness and subtracting the smaller number from the larger number.

It should be understood that while FIG. 3 illustrates a single line of rectangular indicators 35, this is merely an example. As described above, some embodiments may utilize a grid or other configuration of indicators that form a plurality of lines, patterns, etc. Similarly, the rectangular indicators 35 may be integrated into the wetness detection device 20 and/or applied to an exterior surface of the insert 30.

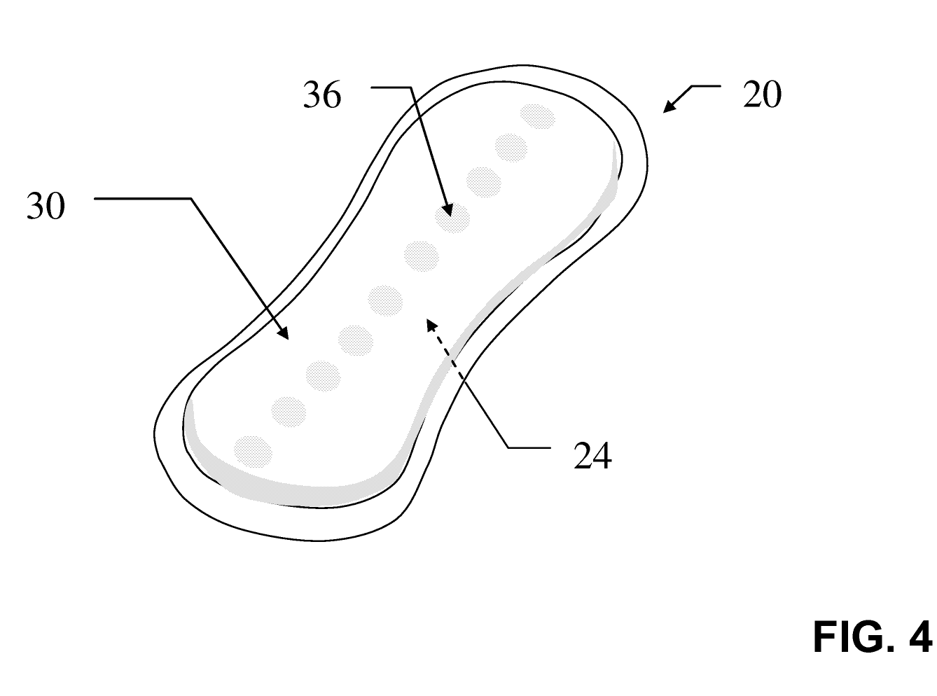

FIG. 4 depicts a wetness detection device 20 for determining a volume of a liquid through a linear measurement of liquid on a plurality of rounded indicators 36, according to embodiments disclosed herein. While the embodiment of FIG. 3 illustrates a plurality of rectangular indicators 35, the rounded indicators 36 may be utilized for purposes of comfort, accuracy, etc. Additionally, as with FIG. 3, the rounded indicators 36 may form any configuration or pattern for detecting the presence and volume of liquid and may be integrated into the wetness detection device 20 and/or applied to an exterior portion of the insert 30.

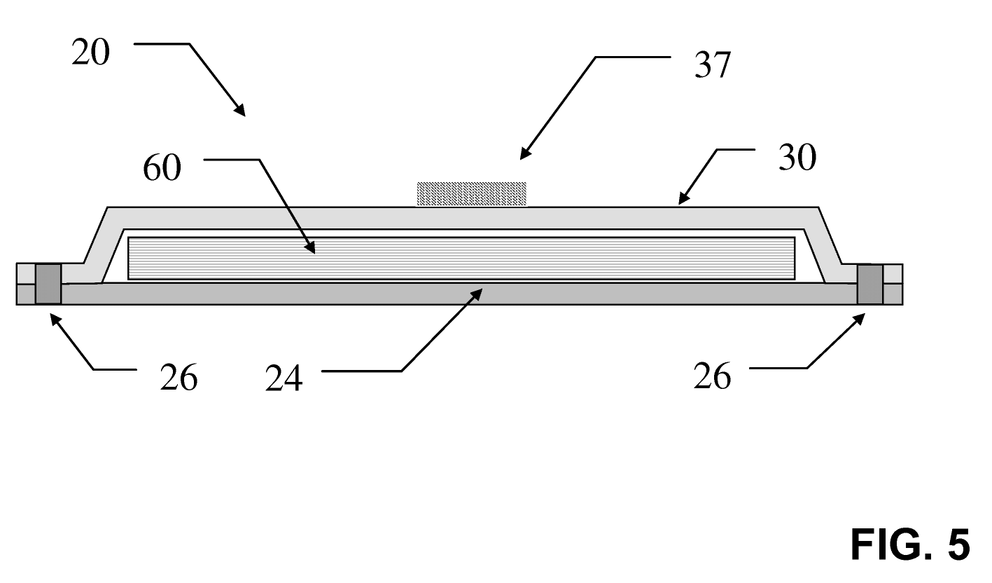

FIG. 5 depicts a cross sectional view of a wetness detection device 20 determining a volume of a liquid through a linear measurement of liquid on an indicator 34 that is applied to an outer surface of the wetness detection device 20, according to embodiments disclosed herein. As illustrated, the wetness detection device 20 may include the backsheet 24, the insert 30, and the securing mechanisms 26, as described above. However, in this embodiment, the indicator 37 is applied to an exterior surface of the insert 30. This allows a wearer to purchase off-the-shelf liquid absorption garments and apply the indicator 37 to the insert 30. Additionally, the wearer may customize the location and orientation of the insert 37, based on the wearer's needs.

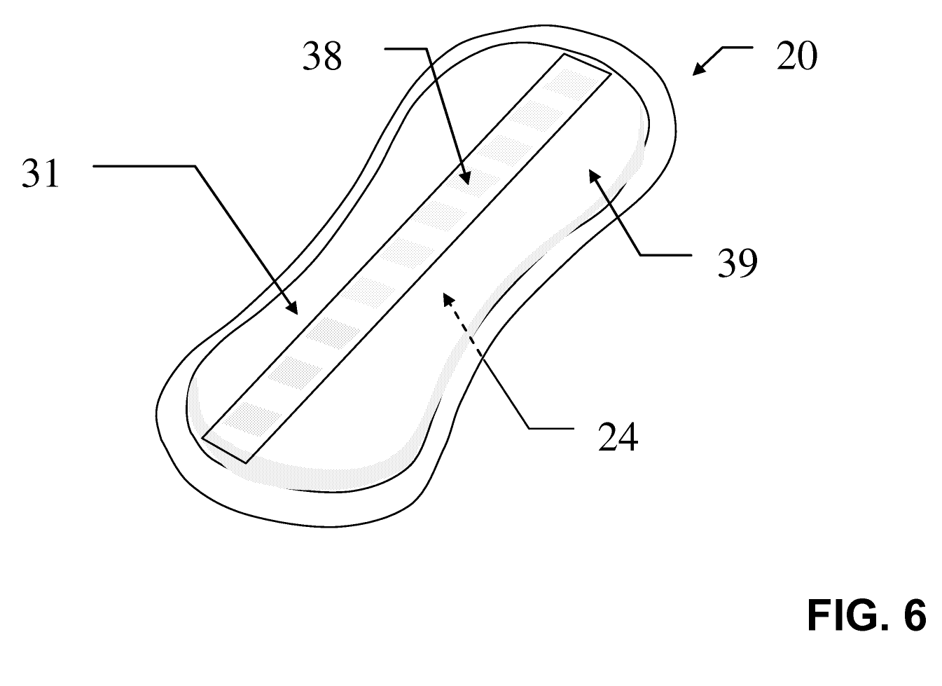

FIG. 6 depicts a wetness detection device 20 for determining a volume of a liquid through a linear measurement of liquid on a continuous indicator applied to the wetness detection device 20, according to embodiments disclosed herein. As illustrated, the wetness detection device 20 may include the backsheet 24, as described above. However, in the example of FIG. 6, the wetness detection device 20 includes an integrated topsheet 39 and an insert 31 that is applied by the wearer (or other user) to the integrated topsheet 39. The insert 31 may include a plurality of indicators 38 for detecting the presence and volume of liquid.

It should be understood that, while the insert 31 is depicted in FIG. 6 as a strip, this is merely an example. Some embodiments are configured with the insert 31 as a grid, circle, oval, pattern, and/or other configured for performing the desired function.

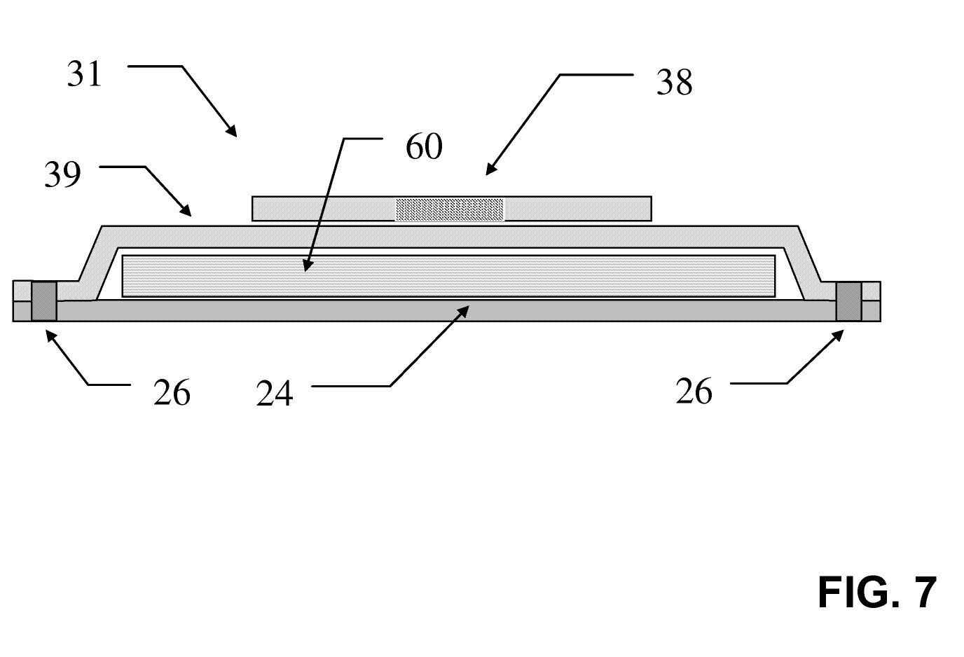

FIG. 7 depicts a cross sectional view of a wetness detection device 20 determining a volume of a liquid through a linear measurement of liquid on an insert 30 of the wetness detection device 20, according to embodiments disclosed herein. As illustrated, the wetness detection device 20 includes the backsheet 24 and the securing mechanisms 26, as described above. However, FIG. 7 depicts an integrated topsheet 39 and an insert 31 that has been applied after manufacture (such as by a wearer or other user). The insert 31 may include adhesive or other coupling mechanism to facilitate the coupling to the topsheet 39. The insert 31 may include one or more indicators 38 for detecting the presence and volume of liquid. As discussed above, this provides the wearer with the freedom to apply wetness detecting capabilities to any type of wetness absorbing garment.

It should be understood that while the embodiments discussed above are directed to a wetness detection device 20, this is merely an example. In some embodiments, the wetness detection device 20 is part of a kit for assessing the strength of a person's pelvic floor muscles that affect urinary incontinence and/or training a wearer to perform pelvic floor exercises. The pelvic floor exercises may include exercises such as KNACK exercises, which develop reflexive response that helps prevent incontinence issues associated with sneezing, coughing, and/or other involuntary responses that exert pressure on the bladder. The kit may additionally include instructions, such as textual instructions, audio instructions, and/or video instructions for testing and/or exercising the pelvic floor muscles, as well as a slender insertion object that may provide direct physical and visual feedback to the wearer (or administrator) while performing the bodily exercise. The instructions may include one or more of the following: a booklet, pamphlet, leaflet, printed instructions on a package, digital media via a CD, DVD, flash drive, internet link, QR code, and/or other reference.

It should be understood that the instructions may describe the exercise or testing that the wearer will perform. As an example, the instructions may provide a recommendation for a training regimen of contracting pelvic floor muscles during intentional increase of abdominal pressure from coughing, sneezing, laughing, hiccupping, tightening, and/or bearing down on the wearer's abdominal muscles. The instructions may additionally provide a recommendation to seek professional medical help if the amount of fluid absorbed by the insert 30 is greater than a predetermined threshold.

Accordingly, the instructions may describe that the wearer should utilize the wetness detection device 20 to identify the volume of liquid prior to a first exercise. The instructions may indicate that the wearer is to perform the exercises (and/or wait a predetermined amount of time) before utilizing a different wetness detection device to determine any progress that the wearer has made with the pelvic floor muscles. Based on the progress and condition of the wearer's pelvic floor muscles, additional exercises may be instructed, and/or wetness detection devices with different absorption qualities may be recommended.

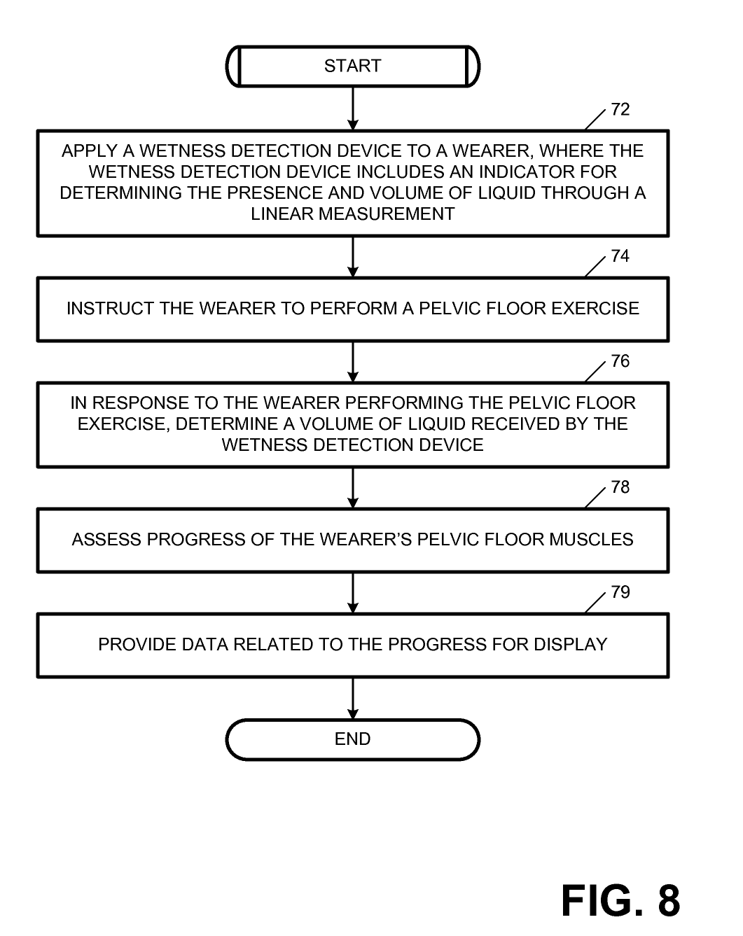

FIG. 8 depicts a flowchart for providing training for pelvic floor muscles for urinary incontinence, according to embodiments disclosed herein. As illustrated in block 72, the wetness detection device 20 may be applied to a wearer, where the wetness detection device 20 includes an indicator for the presence and volume of liquid through a linear measurement. In block 74, the wearer may be instructed to perform a pelvic floor exercise. The instructions may come from a medical professional, electronic device, and/or from the kit that includes the wetness detection device 20. Similarly, the pelvic floor exercise may include testing of the wearer and/or the wearer actually performing exercises to strengthen the pelvic floor muscles. In block 76, in response to the wearer performing the pelvic floor exercise, a volume of liquid received by the wetness detection device 20 may be determined from a linear wetness test on a surface of the wetness detection device 20. In block 78, the progress of the wearer's pelvic floor muscles may be assessed. Again, this may be performed via a visual inspection of the wetness detection device 20, and/or via a signal from the wetness detection device 20 to a computing device that makes this assessment.

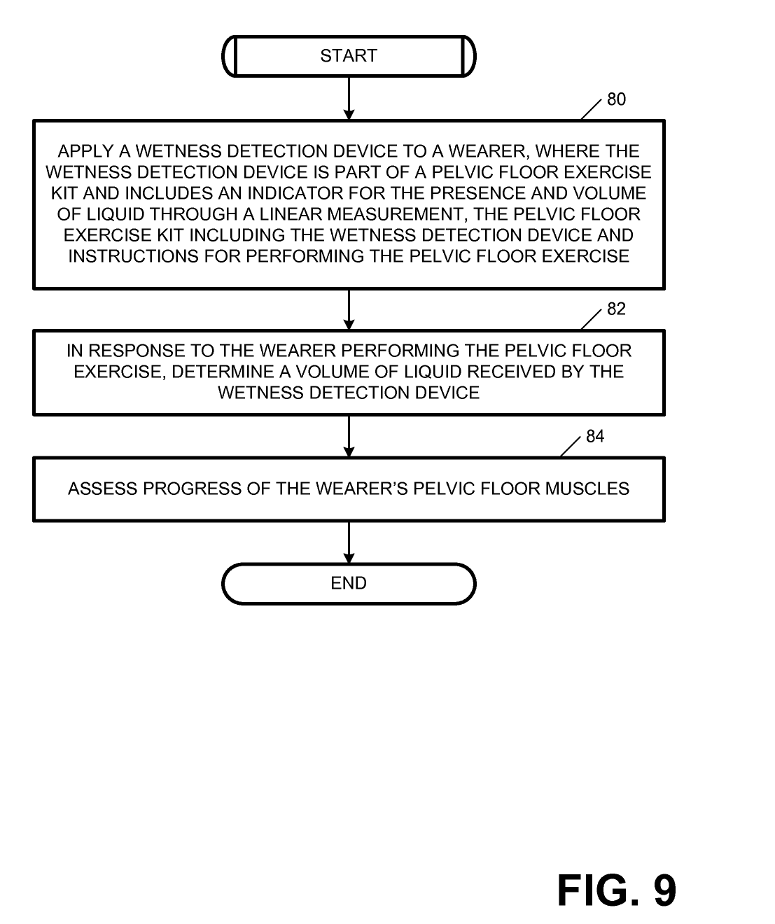

FIG. 9 depicts a flowchart for utilizing a training kit for assessing progress of pelvic floor muscles, according to embodiments disclosed herein. As illustrated in block 80, a wetness detection device 20 may be applied to a wearer. The wetness detection device 20 may be part of a pelvic floor exercise kit and may include an indicator for the presence and volume of liquid through a liner measurement. The pelvic floor exercise kit may include the wetness detection device 20 and instructions for performing the pelvic floor exercise. As discussed above, the instructions may include textual instructions, audio instructions, video instructions, etc. In block 82, in response to the wearer performing the pelvic floor exercise, a volume of the liquid received by the wetness detection device 20 may be determined via a linear measurement of the liquid on the wetness detection device 20. In block 84, the progress of the wearer's pelvic floor muscles may be assessed.

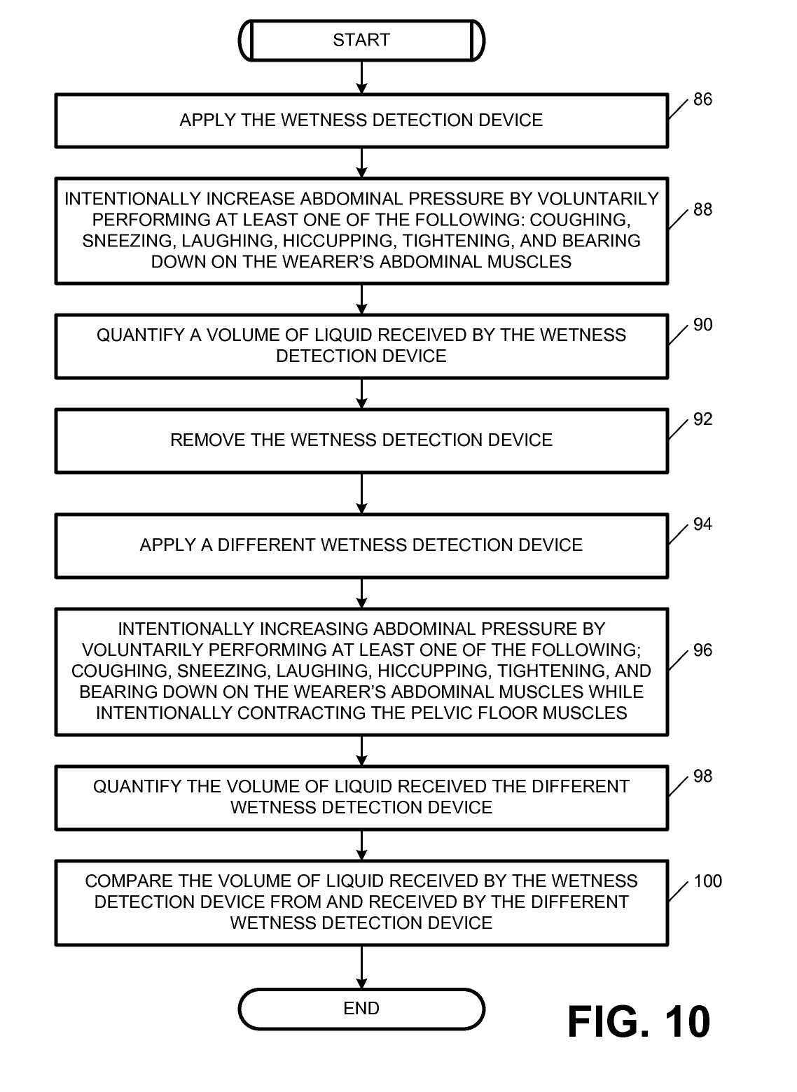

FIG. 10 depicts a flowchart of a process that a wearer may utilize, according to embodiments described herein. As illustrated in block 86, the wetness detection device 20 may be applied to the wearer. In block 88, abdominal pressure may be intentionally increased by the wearer by voluntarily performing at least one of the following: coughing, sneezing, laughing, hiccupping, tightening, and bearing down on the wearer's abdominal muscles. In block 90, a volume of liquid received by the wetness detection device 20 may be quantified. In block 92, the wetness detection device 20 may be removed. In block 94, a different wetness detection device 20 may be applied to the wearer. In block 96, abdominal pressure may be intentionally increased by the wearer by voluntarily performing at least one of the following; coughing, sneezing, laughing, hiccupping, tightening, and bearing down on the wearer's abdominal muscles while intentionally contracting the pelvic floor muscles. In block 98, the volume of liquid received the different wetness detection device 20 may be quantified. In block 100, the volume of liquid received by the wetness detection device 20 from and received by the different wetness detection device 20 may be compared to determined progress of the pelvic floor muscles.

The figures of the present disclosure are intended to illustrate elements, their parts, and their relationships, as described in the specification; the figures are not intended to illustrate any particular relative or absolute size or dimension, unless otherwise stated in the text.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### APPENDIX

In addition, the following references are incorporated by reference in their entireties:

U.S. Pat. No. 4,022,211, entitled “Wetness indicator for absorbent pads” issued on May 10, 1977 to Timmons, et al.

U.S. Pat. No. 4,231,370, entitled “Disposable diaper type garment having wetness indicator” issued on Nov. 4, 1980 to Mroz, et al.

U.S. Pat. No. 4,327,731, entitled “Moisture indicator” issued on May 4, 1982 to Powell.

U.S. Pat. No. 4,681,576, entitled “Wetness indicating hot-melt adhesive” issued on Jul. 21, 1987 to Colon, et al.

U.S. Pat. No. 4,705,513, entitled “Disposable diaper with wetness indicator” issued on Nov. 10, 1987 to Sheldon, et al.

U.S. Pat. No. 4,738,674, entitled “Moisture indicator apparatus and method” issued on Apr. 19, 1988 to Todd, et al.

U.S. Pat. No. 4,743,238, entitled “Wetness indicating hot-melt adhesive” issued on May 10, 1988 to Colon et al.

U.S. Pat. No. 4,895,567, entitled “Wetness indicating hot-melt adhesive” issued on Jan. 23, 1990 to Colon et al.

U.S. Pat. No. 4,931,051, entitled “Wetness indicator” issued on Jun. 5, 1990 to Castello.

U.S. Pat. No. 5,035,691, entitled “Hot melt moisture indicator material for disposable articles” issued on Jul. 30, 1991 to Zimmel, et al.

U.S. Pat. No. 5,066,711, entitled “Wetness indicating hot-melt adhesive” issued on Nov. 19, 1991 to Colon et al.

U.S. Pat. No. 5,089,548, entitled “Hot melt moisture indicator material for disposable articles” issued on Feb. 18, 1992 to Zimmel, et al.

U.S. Pat. No. 5,167,652, entitled “Moisture sensitive film” issued on Dec. 1, 1992 to Mueller.

U.S. Pat. No. 5,342,861, entitled “Hot melt wetness indicator” issued on Aug. 30, 1994 to Raykovitz.

U.S. Pat. No. 5,354,289 entitled “Absorbent product including super absorbent material and fluid absorption capacity monitor” issued on Oct. 11, 1994 to Mitchell, et al.

WO1991019471, entitled “Capacity indicia for absorbent articles” issued on Nov. 1, 1994 to Osborne, et al.

U.S. Pat. No. 5,647,863, entitled “Absorbent article with clean appearance and capacity signal means” issued on Jul. 15, 1997 to Hammons, et al.

U.S. Pat. No. 5,690,624, entitled “Disposable diaper” issued on Nov. 25, 1997 to Sasaki, et al.

U.S. Pat. No. 5.766,212, entitled “Disposable diaper” issued on Jun. 16, 1998 to Jitoe, et al.

U.S. Pat. No. 6,075,178, entitled “Absorbent article with wetness indicator” issued on Jun. 13, 2000.

U.S. Pat. No. 6,515,194, entitled “Diaper having centrally-located chromatographic layer with peripherally-located wetness indicator” issued on Feb. 4, 2003 to Neading, et al.

U.S. Pat. No. 6,596,918, entitled “Absorbent articles having wetness indicating graphics and employing masking techniques” issued on Jul. 22, 2003 to Wehrle, et al.

U.S. Pat. No. 6,653,522, entitled “Hot melt adhesives based on sulfonated polyesters comprising wetness indicator” issued on Nov. 25, 2003 to Blumenthal, et al.

U.S. Pat. No. 6,772,708, entitled “Wetness indicator having improved colorant retention” issued on Aug. 10, 1994 to Klofta, et al.

U.S. Pat. No. 6,904,865, entitled “Wetness indicator having improved colorant retention and durability” issued on Jun. 14, 2005 to Klofta, et al.

U.S. Pat. No. 7,159,532, entitled “Wetness indicator having improved colorant retention and durability” issued on Jan. 9, 2007 to Klofta, et al.

U.S. Pat. No. 7,172,667, entitled “System and method for incorporating graphics into absorbent articles” issued on Feb. 6, 2007 to Vergona.

U.S. Pat. No. 7,178,571, entitled “System and method for incorporating graphics into absorbent articles” issued on Feb. 20, 2007 to Vergona.

U.S. Pat. No. 7,306,764, entitled “Wetness indicator” issued on Dec. 11, 2007 to Mody.

U.S. Pat. No. 7,332,642, entitled “Disposable absorbent articles having printed wetness indicators” issued on Feb. 19, 2008 to Liu, each of which is incorporated herein by reference.

U.S. Pat. No. 5,389,094 entitled “Capacity indicia for absorbent articles” issued to Lavash, et al. on Feb. 14, 1995.

U.S. Pat. No. 5,558,663 entitled “Absorbent articles having undergarment covering components with zones of extensibility” issued to Weinberger, et al. on Sep. 24, 1996.

U.S. Pat. No. 4,950,264 entitled “Thin, flexible sanitary napkin” issued to Osborn on Aug. 21, 1990.

U.S. Pat. No. 5,009,653 entitled “Thin, flexible sanitary napkin” issued to Osborn on Apr. 23, 1991.

Non-Patent Literature entitled “A pelvic muscle precontraction can reduce cough-related urine loss in selected women with mild SUI”, J. Am. Geriatr. Soc., 46, 870-874 (1998), by Janis M. Miller, James A. Ashton-Miller, and John O. L. DeLancey.

What is claimed is:

1. A wetness detection device for identifying a condition of pelvic floor muscles, comprising:
a backsheet, wherein the backsheet is substantially impervious to liquid;
an insert that is coupled to the backsheet via a securing mechanism, wherein the insert is constructed with a predetermined absorbency; and
an indicator that is coupled to the insert, wherein the indicator reacts when in contact with the liquid, such that because of the predetermined absorbency of the insert, the indicator communicates an amount of fluid leaked during contraction of abdominal muscles.

2. The wetness detection device of claim 1, wherein the insert is configured according to at least one of the following: incorporated within the wetness detection device and coupled to an exterior surface of the wetness detection device.

3. The wetness detection device of claim 1, wherein the wetness detection device includes at least one of the following: a liner, a menstrual pad, and an incontinence product.

4. The wetness detection device of claim 1, wherein the insert includes a nonwoven material that is configured according to at least one of the following: hydrophilic or rendered hydrophilic.

5. The wetness detection device of claim 1, wherein the insert is configured as a top sheet.

6. The wetness detection device of claim 1, wherein the insert includes a primary top sheet and a secondary top sheet.

7. The wetness detection device of claim 1, wherein the indicator performs at least one of the following in response to detecting the liquid: change color, change visibility, and send an electrical signal.

8. The wetness detection device of claim 1, further comprising a plurality of indicators configured in a pattern for quantifying a volume of liquid absorbed by the insert based on at least one of the following: length of sprawl of the liquid and area of sprawl of the liquid.

9. A kit for identifying a condition of pelvic floor muscles, comprising:
a wetness detection device, comprising:
a backsheet, wherein the backsheet is substantially impervious to liquid;
an insert that is coupled to the backsheet via a securing mechanism, wherein the insert is constructed with a predetermined absorbency; and
an indicator that is coupled to the insert, wherein the indicator reacts when in contact with the liquid, such that because of the predetermined absorbency of the insert, the indicator identifies progress on pelvic floor muscles of a wearer of the wetness detection device; and
instructions for exercising the pelvic floor muscles to perform while wearing the wetness detection device, wherein the instructions describe application of the wetness detection device.

10. The kit of claim 9, wherein exercising the pelvic floor muscles includes applying downward pressure on the wearer's abdomen while the wearer intentionally performs at least one of the following: cough, sneeze, laugh, hiccup, tighten, and bearing down the wearer's abdominal muscles.

11. The kit of claim 9, wherein the instructions further describe at least one of the following:
applying the wetness detection device;
intentionally increasing abdominal pressure by voluntarily performing at least one of the following: coughing, sneezing, laughing, hiccupping, tightening, and bearing down on the wearer's abdominal muscles;
quantifying a volume of liquid received by the wetness detection device;
removing the wetness detection device;
applying a different wetness detection device;
intentionally increasing abdominal pressure by voluntarily performing at least one of the following; coughing, sneezing, laughing, hiccupping, tightening, and bearing down on the wearer's abdominal muscles while intentionally contracting the pelvic floor muscles;
quantifying the volume of liquid received by the different wetness detection device; and
comparing the volume of liquid received by the wetness detection device and received by the different wetness detection device.

12. The kit of claim 9, wherein the instructions are communicated to a user according to at least one of the following formats: textual, visual, and audio.

13. The kit of claim 9, wherein the instructions also provide a recommendation to seek professional medical help if a volume of liquid received by the insert exceeds a predetermined threshold.

14. The kit of claim 9, wherein the insert is configured according to at least one of the following: incorporated within the wetness detection device and coupled to an exterior surface of the wetness detection device.

15. The kit of claim 9, wherein the wetness detection device includes at least one of the following: a liner, a menstrual pad, and an incontinence product.

16. A method for identifying a condition of pelvic floor muscles comprising:
applying a wetness detection device to a wearer, where the wetness detection device includes an indicator for detecting presence and a volume of liquid through a linear measurement;
instruct the wearer to perform a pelvic floor exercise;
in response to the wearer performing the pelvic floor exercise, determining the volume of liquid received by the wetness detection device;
assessing progress of the wearer's pelvic floor muscles based on an indication from the wetness detection device; and
providing data related to the progress for display.

17. The method of claim 16, wherein assessing progress of the wearer's pelvic floor muscles is provided by a circuit that is coupled to the wetness detection device.

18. The method of claim 16, wherein the indicator performs at least one of the following in response to detecting liquid: change color, change visibility, and send an electrical signal.

19. The method of claim 16, wherein method is performed according to instructions provided in a kit with the wetness detection device.

20. The method of claim 19, wherein the instructions are communicated to a user according to at least one of the following formats: textual, visual, and audio.
